(19) United States
(12) Patent Application Publication
(10) Pub. No.: US 2004/0248869 A1
(43) Pub. Date: Dec. 9, 2004

(54) Method for finding novel active ingredients having androgen-type properties

(76) Inventors: Gilles Pauly, Nancy (FR); Veronique Gillon, Essey-les-Nancy (FR); Jean-Luc Contet-Audonneau, Saint-Max (FR); Louis Danoux, Saulxures les Nancy (FR)

Correspondence Address: COGNIS CORPORATION, PATENT DEPARTMENT, 300 BROOKSIDE AVENUE, AMBLER, PA, 19002, US

(21) Appl. No.: 10/491,487

(22) PCT Filed: Sep. 23, 2002
(86) PCT No.: PCT/EP02/10666

(30) Foreign Application Priority Data
Oct. 1, 2001 (EP) 01402527.4

(51) Int. Cl. C12Q 1/00; A61K 31/56

(52) U.S. Cl. 514/177; 435/4

(57) ABSTRACT

Processes for evaluating androgenic properties of various compounds and/or compositions are described, wherein the processes comprise: (a) providing a culture of at least one male fibroblast, the at least one male fibroblast having a metabolic activity; (b) contacting the culture with a potential active component; and (c) determining the metabolic activity of the fibroblast against a standard.

### FIELD OF THE INVENTION

This invention relates generally to men's cosmetics and, more particularly, to a process by which the effectiveness of potential active components can be evaluated and to the use of male fibroblasts in this process.

### PRIOR ART

The human skin is the scene for a number of metabolic reactions involving androgens. Typical examples include the 5-α-reduction of testosterone (T) to the active metabolite dihydrotestosterone (DHT), the 3-α-reduction of DHT, the conversion of weak androgens, such as dehydroepiandrosterone and Δ4-androsteronedione for example, to biologically more active steroids, or the intracellular binding of androgens to specific receptor proteins and the nuclear translocation of androgen receptors. Hair follicles, sebaceous glands and apocrine glands contain particularly high concentrations of androgens. These can stimulate hair growth on certain areas of the skin, for example in the axillae. However, they can also promote baldness in genetically predisposed males. Androgens can also influence the size and the secretion of glands and can thus adversely affect the appearance of the skin, for example by causing acne or seborrhoea. The localization of androgen receptors in the genital fibroblasts of human skin is described, for example, by Liang et al. in J. Invest. Dermatol. 100, 663-666 (1993). The effect of androgens on the growth of genital fibroblasts is normally determined by flow cytometry. It is known from studies conducted by Loire et al. [Biol. of the cell 56, 8989-1992 (1986)] and Sultan et al. [Brit. J. Dermatol. 107, 40-46 (1982)] that dihydrotestosterone does not affect the DNA or protein distribution in the target cell. This suggests that androgens do not induce overall protein synthesis or DNA production. Investigation of the influence of testosterone on the differentiation of human skin cells has shown that testosterone does not influence the volume of the epidermis tissue or the cell size or the number of keratin filaments, but does counteract the reduction of granular cells, keratohyalin granules and keratinosomes. This gives an indication of its role in cell differentiation. Kligman et al. report that the topical application of testosterone propionate in the axillae of relatively old males increases the strength of the epidermis [Adv. Biol. Skin, 6, 177-198 (1965)].

Basically, it is known that androgen production in men decreases with age. It is also known that the cell response to hormones changes with time. Thus, it has been shown that the number of binding sites to androgen receptors in the fibroblasts decreases with age. More detailed studies are based on the assumption that these changes are not directly related to the testosterone level, but are the result of a genetic process. This raises the question of why existing knowledge of male hormones should not be used to develop a model for testing the effectiveness of new potential active components for the development of men's cosmetics.

Accordingly, the problem addressed by the present invention was to provide a process for finding new active components with androgen-like activity for the production of cosmetic preparations specifically for use in men's cosmetics.

### DESCRIPTION OF THE INVENTION

The present invention relates to a process for finding new active components with androgen-like properties for the production of cosmetic preparations for men, in which the potential active components are contacted with cultures of male fibroblasts and the metabolism—more especially the quantity of dermal macromolecules formed by the fibroblasts —is determined against a standard in the course of the interaction.

It has surprisingly been found that cultures of male fibroblasts represent ideal systems for investigating the effectiveness of potential new active components with androgenic or androgen-like activity for the development of male cosmetics. The process makes use of the observation that active components, by interacting with the fibroblasts, stimulate the synthesis of dermal macromolecules, such as for example hyaluronic acid, chondroitin sulfate, dermatan sulfate, keratan sulfate, heparan sulfate, collagen or elastin, whereas non-active compounds produce little or no change in the synthesis rate relative to the blank value. Male sex hormones have been successful as standards. Accordingly, by measuring and following the formation of the dermal macromolecules and comparing it with the standard, it is possible to assess the effectiveness of the test products.

Using this process, it has already been possible to show that individual fractions of Grifola frondosa extracts influence the growth and metabolism of male fibroblasts (see Examples). Grifola frondosa (also known as maitake) are edible higher fungi of the genus Basidiomycetes. These are so-called bracket fungi which are also known as basidiomycetes.

Accordingly, the present invention also relates to the use of extracts or fractions of extracts of the fungus Grifola frondosa for stimulating male fibroblasts, more particularly for stimulating and improving cell renewal, and to their use for the production of skin care preparations for men.

The stimulation of male fibroblasts has also been demonstrated for extracts of Pisum sativum and for a preparation containing mannitol, cyclodextrin, yeast extract and disodium succinate.

Carrying Out the Process

Both embryogenic and male adult fibroblasts may be used and cultivated in the process. Fibroblasts in the present context are understood to include any cells and cell types which have fibroblast-like properties, especially receptors at their surface which are comparable or identical with those of the fibroblasts. The fibroblasts may also be used together with other cells or cell lines, but especially skin cells such as, for example, melanocytes, Langerhans cells, endothelial cells, keratinocytes, neurones and the like. The male sex hormones which may serve as standards and against which the activity of the potential active component may be tested and evaluated include, for example, testosterone, dihydrotestosterone, dehydroepiandrosterone (DHEA) and precursors and derivatives thereof. Both the active components and the standards may be used in quantities of 0.0001 to 0.01% by weight, based on the cultures, and are typically used in a quantity of 0.0002% by weight. The choice of the potential active components is not critical insofar as the system functions reliably as long as the potential active components stimulate the synthesis of dermal macromolecules. In practice, the test substances will preferably be selected from the group of sterols, peptides, proteins, lipids, sugars and polysaccharides because they can be expected to provide the greatest stimulation activity. The parameter monitored is the formation of dermal macromolecules from the group of glycosaminoglycans, proteoglycans, glycoproteins and proteins.

The process may be carried out, for example, as follows: cell suspensions of human fibroblasts (embryonal cells of adult male origin) are mixed with 1 to 2 mg/ml of a collagen suspension. The fibroblasts present in the collagen gel are then cultivated together with a nutrient medium (2% by weight foetal calf serum, FCS) in Petri dishes (5 ml/dish) to which 0.0002% by weight of the potential active component or testosterone or dihydrotestosterone is added as standard. After incubation for 7 days at 27° C./5% by volume carbon dioxide in a steam-saturated atmosphere, biopsies are taken and histological sections prepared. The quantity of macromolecules formed can be determined by two different methods:

1. a general determination by coloring the glucosaminoglucans (GAGs) with PAS Alcian Blue (standard coloration) and

2. a specific immunohistochemical characterization of certain elements of the matrix by treatment with antibodies, more especially anti-chondroitin sulfate, anti-keratan sulfate, anti-heparan sulfate, anti-dermatan sulfate, anti-elastin and anti-collagen (type III).

The fibroblasts are examined with a photon microscope in regard to the coloration (PAS Alcian) and with a confocal laser microscope in regard to the immunohistology. In order to determine the quantity of dermal macromolecules formed, micrographs are taken and the zones around the fibroblasts (“perifibroblastic zones”) are analyzed. Counting is done by image analysis, i.e. by determining the perifibroblastic zones and the gray level. Both parameters are directly proportional to the synthesis of dermal macromolecules. The results are evaluated in relation to the corresponding results without addition of the active components.

Commercial Applications

The synthesis of the dermal macromolecules is, quite generally, a quantitative indication of how age and environmental influences impair the processes in human skin. Accordingly, the present invention relates not only to the use of male fibroblasts on their own or together with other skin cells in the above-described process for finding new active components with androgen-like properties for the production of cosmetic preparations for men, but also to their use—again on their own or together with other cells or cell lines, but especially skin cells—

for investigating pathological effects on the stimulation of cell synthesis;

for investigating the effects of ageing on the stimulation of cell synthesis;

for investigating the effects of stress on the stimulation of cell synthesis and

for investigating the action mechanism of the stimulation of the synthesis of dermal macromolecules by male sex hormones.

### EXAMPLES

As described under the heading “Carrying Out the Process”, various fibroblast cultures were cultivated with and without added male sex hormones and the synthesis of the dermal macromolecules was investigated. The results are set out in Table 1. The numbers represent the increase in %-rel., based on the blank value, i.e. the absence of the male sex hormones, and expressed as the average of the two measuring conditions.

TABLE 1
Synthesis of dermal macromolecules

| | 1 | 2 | 3 | 4 | 5 | 6 |
|---|---|---|---|---|---|---|
| Hormone type | T (soluble) | DHT | T | DHT | T | DHT |
| Fibroblasts | E | MA | MA | MA | MA | MA |
| Increase in the | | | | | | |
| synthesis of | | | | | | |
| macromolecules | | | | | | |
| Chondroitin | +23 | | | +132 | +537 | +79 |
| sulfate | | | | | | |
| Keratan sulfate | | | | | +83 | |
| Heparan sulfate | | +486 | | | | |
| Collagen | +17 | | +113 | +1742 | +654 | +409 |
| Elastin | +11 | | | | +2753 | +552 |

T = testosterone
E = embryonal fibroblasts
DHT = dihydrotestosterone
MA = male adult fibroblasts

### Example 2

Preparation of the Grifola Frondosa (Maitake) Extract with Aqueous Ethanol

120 g dried Granola fronds fungi were introduced into a glass reactor containing 1.2 liters 96% by weight aqueous ethanol. The infusion was extracted with stirring for 1 hour at boiling temperature. The mixture was then cooled to 20° C. and the supernatant colloidal liquid was separated from the residue by filtration through a depth filter with a mean porosity of 450 nm (from Seitz, Bordeaux/France). The alcohol was then removed under reduced pressure at 45° C. and the residue was dried for 12 hours at 50° C. The yield of dry product was 10.86% by weight, based on the dry weight of fungi used.

Fractionation of the Extract

6.26 g of the extract were eluted with methanol in various concentrations in a chromatography column. Preferred adsorption columns are Amberite or reversed phase C18 silica columns. Molecules with the least affinity for the column were collected in the first washing water step. Water, 20% by vol. methanol, 40% by vol. methanol, 60% by vol. methanol, 80% by vol. methanol and pure methanol were used as stepwise gradients. The eluent was used at a flow rate of 10 ml/min.

The alcohol of the individual fractions obtained was evaporated off and the fractions were then separately freeze-dried.

The fractions of Table 2 were used for the following test.

TABLE 2
Ingredients of the fungus Grifola frondosa
obtained by fractionation

| | | | Yield of dried |
|---|---|---|---|
| | | | fraction (quantity of |
| | | | fraction/quantity |
| Eluent | Fraction | Eluted components | of crude extract) |
| Methanol 60% | A | Phenols, amino acids | 1.69% |
| Methanol 80% | B | Phenols, amino acids | 2.68% |

### Use of the Fractions of the Grifola Frondosa Extract in the Test on in Vitro Cultures of Male Fibroblasts

The in vitro growth test on male fibroblast cultures was carried out to demonstrate possible stimulation of the fibroblasts and the regenerating and growth-promoting properties of Grifola frondosa extracts.

A toxicity test enables the optimal concentration for more specific tests on cell cultures to be determined. The growth test allows the capacity of an active component to improve cell renewal to be determined.

Test Procedure:

Male human fibroblasts were cultivated in a standard cell culture medium (DMEM) containing 10% by weight foetal calf serum (FCS). After incubation for one day at 37° C./5% carbon dioxide, the growth medium was replaced by a standard medium without FCS or by a standard medium containing 1% delipidated FCS to which a defined quantity of the fractions to be investigated dissolved in 1% by vol. ethanol had been added.

After incubation for 3 days, the quantity of cell proteins and ATP in the cell culture was determined.

Results:

TABLE 3a
Determination of the quantity of cell proteins and ATP
in in vitro cell cultures of fibroblasts after incubation with
fractions of the Grifola frondosa extract (without FCS)

Without FCS*

| Fraction | Concentration | Proteins | ATP |
|---|---|---|---|
| A | 0.003 | 113 | 116 |
| | 0.001 | 127 | 125 |
| B | 0.003 | 124 | 119 |
| | 0.001 | 135 | 114 |

*results in % versus control (medium without extract fractions)

TABLE 3b
Determination of the quantity of cell proteins and ATP
in in vitro cell cultures of fibroblasts after incubation with
fractions of the Grifola frondosa extract (with 1%
delipidated FCS)

1% Delipidated FCS**

| Fraction | Concentration | Proteins | ATP |
|---|---|---|---|
| A | 0.003 | 110 | 118 |
| | 0.001 | 109 | 127 |
| B | 0.003 | 118 | 116 |
| | 0.001 | 127 | 114 |

**results in % versus control (1% delipidated FCS-without extract fractions)

The results show that the two selected fractions of the Grifola frondosa extract show clear activity in stimulating the cell growth of human male fibroblasts.

Despite addition of the already nutrient-richer cell culture medium used, the extract fractions show an improvement in ATP and protein rate, even with 1% delipidated FCS, against the control.

1-13. (Canceled)

14: A process for evaluating androgenic properties, said process comprising:
(a) providing a culture of at least one male fibroblast, the at least one male fibroblast having a metabolic activity;
(b) contacting the culture with a potential active component; and
(c) determining the metabolic activity of the fibroblast against a standard.

15: The process according to claim 14, wherein the metabolic activity comprises the quantity of a dermal macromolecule formed by the fibroblast culture.

16: The process according to claim 15, wherein the dermal macromolecule is selected from the group consisting of glycosaminoglycans, proteoglycans, glycoproteins, and proteins.

17: The process according to claim 14, wherein the at least one male fibroblast comprises a human male fibroblast.

18: The process according to claim 14, wherein the at least one male fibroblast comprises a human male dermal fibroblast.

19: The process according to claim 14, wherein the culture further comprises at least one additional cell or cell line.

20: the process according to claim 19, wherein the at least one additional cell or cell line comprises a skin cell selected from the group consisting of melanocytes, Langerhans cells, endothelial cells, keratinocytes, and neurones.

21: The process according to claim 14, wherein the standard comprises a second culture of the at least one male fibroblast and a male sex hormone.

22: The process according to claim 21, wherein the male sex hormone is selected from the group consisting of testosterone, dihydrotestosterone, dehydroepiandrosterone, precursors thereof, derivatives thereof and mixtures thereof.

23: The process according to claim 21, wherein the male sex hormone is combined with the second culture in an amount of from 0.0001 to 0.01% by weight, based on the second culture.

24: The process according to claim 14, wherein the potential active component is combined with the culture in an amount of from 0.0001 to 0.01% by weight, based on the culture.

25: A process for evaluating androgenic properties, said process comprising:
(a) providing a culture of at least one human male dermal fibroblast, the at least one human male dermal fibroblast having a metabolic activity which comprises formation of a dermal macromolecule selected from the group consisting of glycosaminoglycans, proteoglycans, glycoproteins, and proteins;
(b) contacting the culture with a potential active component; and
(c) determining the metabolic activity of the fibroblast against a standard, wherein the standard comprises a second culture of the at least one male fibroblast and a male sex hormone selected from the group consisting of testosterone, dihydrotestosterone, dehydroepiandrosterone, precursors thereof, derivatives thereof and mixtures thereof.

26: The process according to claim 25, wherein the potential active component is combined with the culture in an amount of from 0.0001 to 0.01% by weight, based on the culture.

27: The process according to claim 25, wherein the male sex hormone is combined with the second culture in an amount of from 0.0001 to 0.01% by weight, based on the second culture.
